(19) United States
(12) Patent Application Publication
(10) Pub. No.: US 2003/0032834 A1
(43) Pub. Date: Feb. 13, 2003

(54) Aminoalcohol derivatives

(76) Inventors: Hiroshi Kayakiri, Chuo-ku Osaka-shi (JP); Naoaki Fujii, San Francisco, CA (US); Hitoshi Hamashima, Osaka-shi (JP); Minoru Sakurai, Osaka-shi (JP); Kenichi Washizuka, Osaka-shi (JP); Yasuyo Tomishima, Osaka-shi (JP); Kiyoshi Taniguchi, Osaka-shi (JP); Naoko Unami, Osaka-shi (JP); Yutaka Kono, Osaka-shi (JP); Hirofumi Ishikawa, Osaka-shi (JP); Nobuhiro Yamamoto, Osaka-shi (JP); Hisashi Mimura, Osaka-shi (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 10/203,626

(22) PCT Filed: Feb. 20, 2001
(86) PCT No.: PCT/JP01/01205

(30) Foreign Application Priority Data
Feb. 21, 2000 (AU) PQ 5753

(51) Int. Cl. C07C 271/28; A61K 31/325

(52) U.S. Cl. 560/27; 514/483

(57) ABSTRACT

A compound of the formula [Ig]; wherein R¹ is hydrogen or an amino protective group, R₂ is hydrogen or hydroxy, and R₃ and R₄ are independently N-methyl-methoxycarbonylamino, N-ethyl-methoxycarbonylamino, N-propyl-methoxycarbonylamino or 3-ethylureid, or R³ and R⁴ are both methoxycarbonylamino substituted at a meta position of the benzene rings, or a salt thereof, and (2S)-1-phenoxy-3-[3,3-bis[4-methoxycarbonylamino)-phenyl]propylamino]-2-propanol sulfate (2:1) and some crystal forms thereof as β₃adrenergic receptor agonists.

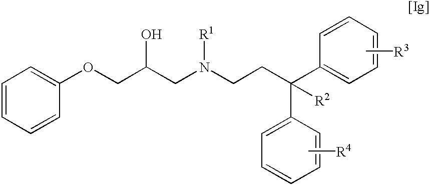

### TECHNICAL FIELD

This invention relates to new aminoalcohol derivatives and salts thereof which are beta-3 (β₃) adrenergic receptor agonists and useful as a medicament.

### DISCLOSURE OF INVENTION

This invention relates to new aminoalcohol derivatives which are β₃ adrenergic receptor agonists, salts thereof and crystal forms thereof.

More particularly, it relates to new aminoalcohol derivatives, salts thereof and crystal forms thereof which have gut sympathomimetic, anti-ulcerous, anti-pancreatitis, lipolytic, anti-urinary incontinence, anti-pollakiuria activities, anti-diabetes and anti-obesity, to processes for the preparation thereof, to a pharmaceutical composition comprising the same and to a method of using the same therapeutically in the treatment and/or prevention of gastro-intestinal disorders caused by smooth muscle contractions in human beings or animal.

One object of this invention is to provide new and useful aminoalcohol derivatives, salts thereof and crystal forms thereof which have gut sympathomimetic, anti-ulcerous, lipolytic, anti-urinary incontinence, anti-pollakiuria activities, anti-diabetes and anti-obesity.

Another object of this invention is to provide processes for the preparation of said aminoalcohol derivatives, salts thereof and crystal forms thereof.

A further object of this invention is to provide a pharmaceutical composition comprising, as an active ingredient, said aminoalcohol derivatives, salts thereof and crystal forms thereof.

Still further object of this invention is to provide a therapeutical method for the treatment and/or prevention of aforesaid diseases in human beings or animals, using said aminoalcohol derivatives, salts thereof and crystal forms thereof.

The object compound of the present invention is the compound of the following formula [Is]:

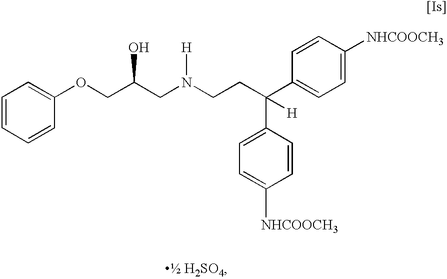

namely, (2S)-1-phenoxy-3-[3,3-bis[4-(methoxycarbonylamino)-phenyl]propylamino]-2-propanol sulfate (2:1) [hereinafter referred to briefly as compound [Is]], or the compound of the following formula [Ifm]:

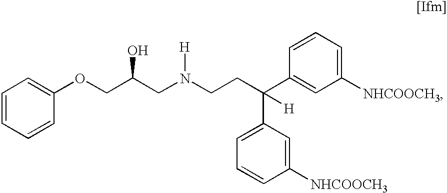

namely, (2S)-1-phenoxy-3-[3,3-bis[3-(methoxycarbonylamino)-phenyl]propylamino]-2-propanol [hereinafter referred to briefly as compound [Ifm]], or a salt thereof.

The other object compound of the present invention can be represented by the following general formula [Ig]:

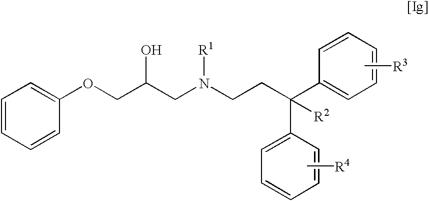

wherein

R¹ is hydrogen or an amino protective group,

R² is hydrogen or hydroxy, and

R³ and R⁴ are independently N-methyl-methoxycarbonylamino, N-ethyl-methoxycarbonylamino, N-propyl-methoxycarbonylamino or 3-ethylureid, or

R³ and R⁴ are both methoxycarbonylamino substituted at a meta position of the benzene rings,

or a salt thereof.

We have already prepared (2S)-1-phenoxy-3-[3,3-bis[4-(methoxycarbonylamino)phenyl]propylamino]-2-propanol hydrochloride (1:1) [hereinafter referred to briefly as compound [Ih]], namely, a salt different from the compound [Is], and have filed an international patent application to cover the compound [Ih] as an application number of PCT/JP99/04538 (cf. Example 30) on Aug. 23, 1999, which was published as WO 00/12462. However, as we could not get the compound [Ih] as crystals and pharmaceutical drugs are in general stabler as crystals than as an amorphous powder, after filing the application, we prepared several different salts thereof and got the compound [Is] as crystals. And, in such our research phase, we found that the compound [Is] exists as two polymorphic forms, i.e. Form A crystallized from a solvent of ethanol as shown in Example 5 mentioned below and Form B crystallized from a solvent of acetone as shown in Example 6 mentioned below.

Further more, in our further research phase, we found that the compound [Is] has a polymorphic form other than Forms A and B aforementioned, i.e. Form D crystallized from a mixed solvent of ethanol, methanol and acetone, or a mixed solvent of methanol and acetonitrile.

According to the present invention, the object compounds can be prepared by processes which are illustrated in the following schemes.

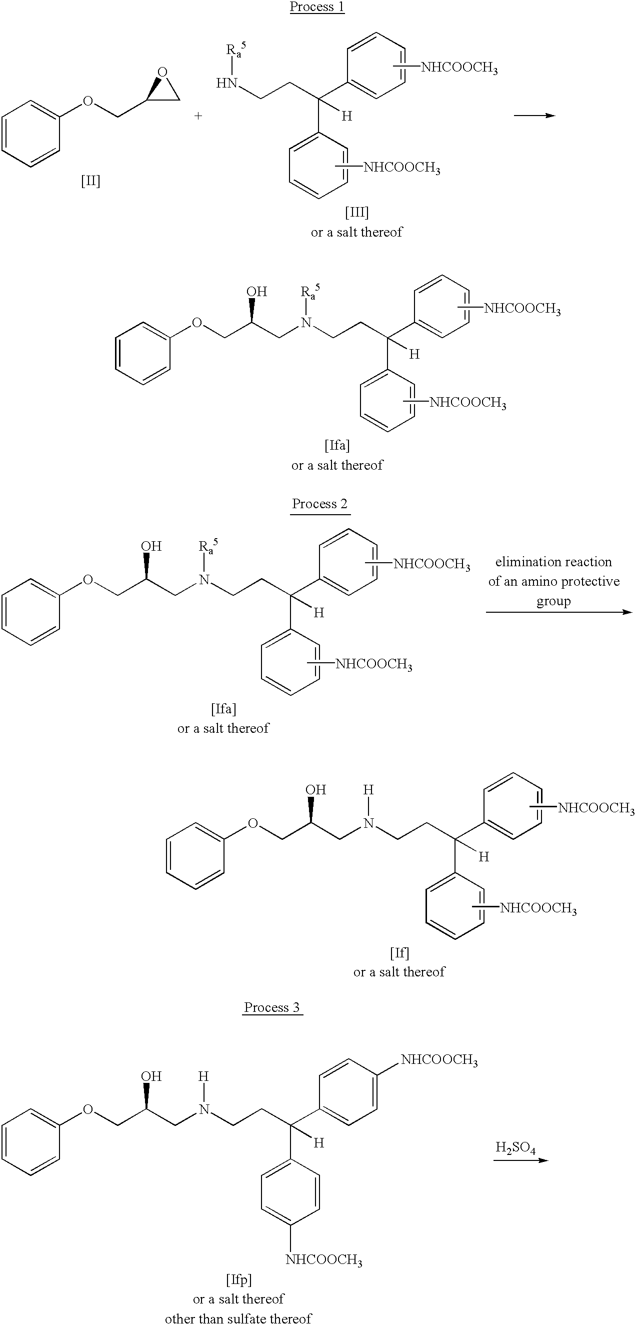

wherein R¹, R², R³ and R⁴ are each as defined above,

Rₐ¹ is an amino protective group, and

Rₐ⁵ is an amino protective group.

In the above and subsequent description of the present specification, suitable example of the various definition to be included within the scope of the invention is explained in detail in the following.

Suitable example of “amino protective group” moiety may be common amino protective group such as acyl, for example, substituted or unsubstituted lower alkanoyl [e.g. formyl, acetyl, propionyl, trifluoroacetyl, etc.], phthaloyl, lower alkoxycarbonyl [e.g. tert-butoxycarbonyl, tert-amyloxycarbonyl, etc.], substituted or unsubstituted aralkyloxycarbonyl [e.g. benzyloxycarbonyl, p-nitrobenzyloxycarbonyl, etc.], substituted or unsubstituted arenesulfonyl [e.g. benzenesulfonyl, tosyl, etc.], nitrophenylsulfenyl, ar(lower)alkyl [e.g. trityl, benzyl, etc.], and the like, in which preferable one is phenyl(lower)alkyl such as benzyl.

Suitable salts of the object aminoalcohol derivatives [Ifm] and [Ig] are pharmaceutically acceptable salts and include conventional non-toxic salts such as an inorganic acid addition salt [e.g. hydrochloride, hydrobromide, sulfate, phosphate, etc.], an organic acid addition salt [e.g. formate, acetate, trifluoroacetate, oxalate, maleate, fumarate, tartarate, citrate, methanesulfonate, benzenesulfonate, toluenesulfonate, etc.] or the like.

The Processes 1 to 5 for preparing the object compounds of the present invention are explained in detail in the following.

Process 1

The object compound [Ifa] or a salt thereof can be prepared by reacting a compound [II] with a compound [III] or a salt thereof.

Suitable salt of the compound [III] may be the same as those exemplified for the compound [Ig].

The reaction is preferably carried out in the presence of a base such as an alkali metal carbonate [e.g. sodium carbonate, potassium carbonate, etc.], an alkaline earth metal carbonate [e.g. magnesium carbonate, calcium carbonate, etc.], an alkali metal bicarbonate [e.g. sodium bicarbonate, potassium bicarbonate, etc.], tri(lower)alkylamine [e.g. trimethylamine, triethylamine, etc.], picoline or the like.

The reaction is usually carried out in a conventional solvent, such as an alcohol [e.g. methanol, ethanol, propanol, isopropanol, etc.], diethyl ether, tetrahydrofuran, dioxane, or any other organic solvent which does not adversely influence the reaction.

The reaction temperature is not critical, and the reaction can be carried out under cooling to heating.

Process 2

The object compound [If] or a salt thereof can be prepared by subjecting a compound [Ifa] or a salt thereof to elimination reaction of the amino protective group.

Suitable salts of the compounds [If] and [Ifa] may be the same as those exemplified for the compound [Ig].

This reaction is carried out in accordance with a conventional method such as hydrolysis, reduction or the like.

The hydrolysis is preferably carried out in the presence of a base or an acid including Lewis acid.

Suitable base may include an inorganic base and an organic base such as an alkali metal [e.g. sodium, potassium, etc.], an alkaline earth metal [e.g. magnesium, calcium, etc.], the hydroxide or carbonate or bicarbonate thereof, hydrazine, trialkylamine [e.g. trimethylamine, triethylamine, etc.], picoline, 1,5-diazabicyclo[4.3.0]non-5-ene, 1,4-diazabicyclo[2.2.2]octane, 1,8-diazabicyclo-[5.4.0]undec-7-ene, or the like.

Suitable acid may include an organic acid [e.g. formic acid, acetic acid, propionic acid, trichloroacetic acid, trifluoroacetic acid, etc.], an inorganic acid [e.g. hydrochloric acid, hydrobromic acid, sulfuric acid, hydrogen chloride, hydrogen bromide, hydrogen fluoride, etc.] and an acid addition salt compound [e.g. pyridine hydrochloride, etc.].

The elimination using trihaloacetic acid [e.g. trichloroacetic acid, trifluoroacetic acid, etc.] or the like is preferably carried out in the presence of cation trapping agents [e.g. anisole, phenol, etc.].

The reaction is usually carried out in a solvent such as water, an alcohol [e.g. methanol, ethanol, etc.], methylene chloride, chloroform, tetrachloromethane, tetrahydrofuran, a mixture thereof or any other solvent which does not adversely influence the reaction. A liquid base or acid can also be used as the solvent. The reaction temperature is not critical and the reaction is usually carried out under cooling to heating.

The reduction method applicable for the elimination reaction may include chemical reduction and catalytic reduction.

Suitable reducing agents to be used in chemical reduction are a combination of metal [e.g. tin, zinc, iron, etc.] or metallic compound [e.g. chromium chloride, chromium acetate, etc.] and an organic or inorganic acid [e.g. formic acid, acetic acid, propionic acid, trifluoroacetic acid, p-toluenesulfonic acid, hydrochloric acid, hydrobromic acid, etc.].

Suitable catalysts to be used in catalytic reduction are conventional ones such as platinum catalysts [e.g. platinum plate, spongy platinum, platinum black, colloidal platinum, platinum oxide, platinum wire, etc.], palladium catalysts [e.g. spongy palladium, palladium black, palladium oxide, palladium on carbon, colloidal palladium, palladium on barium sulfate, palladium on barium carbonate, etc.], nickel catalysts [e.g. reduced nickel, nickel oxide, Raney nickel, etc.], cobalt catalysts [e.g. reduced cobalt, Raney cobalt, etc.], iron catalysts [e.g. reduced iron, Raney iron, etc.], copper catalysts [e.g. reduced copper, Raney copper, Ullman copper, etc.] and the like.

In case that the amino protective group is benzyl, the reduction is preferably carried out in the presence of a combination of palladium catalysts [e.g. palladium black, palladium on carbon, etc.] and formic acid or its salt [e.g. ammonium formate, etc.].

The reduction is usually carried out in a conventional solvent which does not adversely influence the reaction such as water, an alcohol [e.g. methanol, ethanol, propanol, etc.], chlorobenzene, N,N-dimethylformamide, or a mixture thereof. Additionally, in case that the above-mentioned acids to be used in chemical reduction are in liquid, they can also be used as a solvent. Further, a suitable solvent to be used in catalytic reduction may be the above-mentioned solvent, and other conventional solvent such as diethyl ether, dioxane, tetrahydrofuran, etc. or a mixture thereof.

The reaction temperature of this reduction is not critical and the reaction is usually carried out under cooling to heating.

Process 3

The object compound [Is] can be prepared by reacting the compound [Ifp] or a salt thereof other than sulfuric acid salt thereof with sulfuric acid.

The reaction is usually carried out in a conventional solvent such as water, alcohol [e.g. methanol, ethanol, etc.], acetone, 2-butanone, dioxane, acetonitrile, chloroform, methylene chloride, ethylene chloride, tetrahydrofuran, ethyl acetate, N,N-dimethylformamide, pyridine or any other organic solvent which does not adversely influence the reaction, or the mixture thereof, preferably ethanol or acetone.

The reaction temperature is not critical, and the reaction is usually carried out under cooling to warming.

For example, the object compound [Is] can be prepared by the method of Example 5 mentioned below.

Crystals of the object compound [Is] can be obtained

(1) in a process of crystallization of it which is carried out by converting its free base (the compound [Ifp]) to it (semisulfate) in a conventional solvent using sulfuric acid as described in Process 3 mentioned above (for example, the crystal Form A can be obtained as described in Example 5 mentioned below),

(2) in a process of crystallization of it which is carried out by stating from a hot solution of it in a conventional solvent and cooling the solution (for example, the crystal Form D, which has the characteristic diffraction angles 2θ (°) of about 6.41, about 9.70, about 16.85, about 17.93, about 20.82 and about 22.25 as shown in the FIG. 5 of the X-ray powder diffractometry pattern, can be obtained using a mixed solvent of ethanol, methanol and acetone, or a mixed solvent of methanol and acetonitrile as a solvent of the hot solution),

(3) in a process of crystallization of it which is carried out by starting from a solution of an oily, powder or amorphous compound [Is] in a conventional solvent and standing the solution under stirring (for example, the crystal Form B can be obtained as described in Example 6 mentioned below), or

(4) in a process of crystallization of it which is carried out by starting from a solution of it in a conventional good solvent (e.g. methanol, etc.) and adding a conventional poor solvent (e.g. acetone, ethanol, isopropanol, etc.) to the solution.

Process 4

The object compound [Ig] or a salt thereof can be prepared by reacting a compound [II] with a compound [IV] or a salt thereof.

Suitable salt of the compound [IV] may be the same as those exemplified for the compound [Ig].

This reaction can be carried out in a similar manner to that of the aforementioned Process 1, and therefore the reagents to be used and the reaction conditions (e.g., solvent, reaction temperature, etc.) can be referred to those of the Process 1.

Process 5

The object compound [Igb] or a salt thereof can be prepared by subjecting a compound [Iga] or a salt thereof to elimination reaction of the amino protection group.

Suitable salts of the compounds [Iga] and [Igb] may be the same as those exemplified for the compound [Ig].

This elimination reaction can be carried out in a similar manner to that of the aforementioned Process 2, and therefore the reagents to be used and the reaction conditions (e.g., solvent, reaction temperature, etc.) can be referred to those of the Process 2.

The compounds obtained by the above processes can be isolated and purified by a conventional method such as pulverization, recrystallization, column chromatography, reprecipitation, or the like, and converted to the desired salt in conventional manners, if necessary.

It is to be noted that the compound [Ig] and the other compounds may include one or more stereoisomers due to asymmetric carbon atoms, and all of such isomers and mixture thereof are included within the scope of this invention.

It is further to be noted that isomerization or rearrangement of the object compound [Ig] may occur due to the effect of the light, acid, base or the like, and the compound obtained as the result of said isomerization or rearrangement is also included within the scope of the present invention.

It is also to be noted that the solvating forms of the compounds [Is], [Ifm] and [Ig] (e.g. hydrate, acetone solvate, etc.) and any forms of the crystal of the compounds [Is], [Ifm] and [Ig] are included within the scope of the present invention.

The object compounds [Is], [Ifm] and [Ig] or a salt thereof possess gut sympathomimetic, anti-ulcerous, anti-pancreatitis, lipolytic, anti-urinary incontinence and anti-pollakiuria activities, and are useful for the treatment and/or prevention of gastro-intestinal disorders caused by smooth muscle contractions in human beings or animals, and more particularly for the treatment and/or prevention of spasm or hyperanakinesia in case of irritable bowel syndrome, gastritis, gastric ulcer, duodenal ulcer, enteritis, cholecystopathy, cholangitis, urinary calculus and the like; for the treatment and/or prevention of ulcer such as gastric ulcer, duodenal ulcer, peptic ulcer, ulcer caused by non steroidal anti-inflammatory drugs, or the like; for the treatment and/or prevention of dysuria such as pollakiuria, urinary incontinence or the like in case of nervous pollakiuria, neurogenic bladder dysfunction, nocturia, unstable bladder, cystospasm, chronic cystitis, chronic prostatitis, prostatic hypertrophy or the like; for the treatment and/or prevention of pancreatitis, obesity, diabetes, glycosuria, hyperlipidemia, hypertension, atherosclerosis, glaucoma, melancholia, depression or the like; for the treatment and/or prevention of diseases as the result of insulin resistance (e.g. hypertension, hyperinsulinemia, etc.); for the treatment and/or prevention of neurogenetic inflammation; and for reducing a wasting condition, and the like.

Additionally, β₃ adrenergic receptor agonists are known to lower triglyceride and cholesterol levels and to raise high density lipoprotein levels in mammals (U.S. Pat. No. 5,451,677). Accordingly, the object compounds [Is], [Ifm] and [Ig] are useful in the treatment and/or prevention of conditions such as hyper-triglyceridaemia, hypercholesterolaemia and in lowering high density lipoprotein levels as well as in the treatment of atherosclerotic and cardiovascular diseases and related conditions.

In order to show the usefulness of the compounds [Is], [Ifm] and [Ig] for the prophylactic and therapeutic treatment of above-mentioned disease in human beings or animals, the pharmacological test data of a representative compound thereof are shown in the following.

Test

Effect on the increase in intravesical pressure induced by carbachol in anesthetized dog

Test Compound

(1) (2S)-1-Phenoxy-3-[3,3-bis[4-(methoxycarbonylamino)-phenyl]propylamino]-2-propanol sulfate (2:1) (crystal Form A)

Test Method

Female Beagle dogs weighing 8.0-15.0 kg were fasted for 24 hours and maintained under halothane anesthesia. A 12F Foley catheter was lubricated with water soluble jelly, inserted into the urethral orifice and advanced approximately 10 cm until the balloon tip was placed well inside the bladder. The balloon was then inflated with 5 ml of room air and catheter slowly withdrawn just part the first resistance that is felt at the bladder neck. Urine was completely drained out through the catheter, and 30 ml of biological saline was infused. The catheter was connected to pressure transducer, and intravesical pressure was continuously recorded. The test compound was injected intravenously at 5 minutes before the administration of carbachol (1.8 μg/kg).

Test Results

| Treatment | Increase in intravesical pressure (mmHg) |
|---|---|
| Before | 6.2 ± 1.1 |
| Test Compound (1) | 4.9 ± 0.9* |
| (0.01 mg/kg) | |

*P < 0.01 vs Before (ANOVA) (N = 5)

2S-1-Phenoxy-3-[3,3-bis[4-(methoxycarbonylamino)-phenyl]propylamino]-2-propanol sulfate (2:1) (compound [Is]) exists as two polymorphic forms, which are designated Form A (Example 5) and Form B (Example 6). These crystal forms can be characterized by X-ray powder diffractometry (XPD) and differential scanning calorimetry (DSC).

The 2θ values of X-ray diffraction peak for Form A are listed in the following table. The characteristic diffraction angles of Form A were about 6.51°, about 13.80°, about 16.97°, about 19.81°, about 21.95° and about 24.56°.

| Angle 2θ | Relative Intensity |
|---|---|
| (°) | (%) |
| 6.51 | 67 |
| 10.95 | 11 |
| 12.97 | 34 |
| 13.80 | 35 |
| 14.91 | 17 |
| 16.15 | 11 |
| 16.97 | 39 |
| 17.83 | 36 |
| 19.27 | 58 |
| 19.81 | 100 |
| 20.69 | 39 |
| 21.28 | 29 |
| 21.95 | 51 |
| 23.49 | 33 |
| 24.11 | 34 |
| 24.56 | 43 |
| 26.12 | 15 |
| 27.07 | 15 |

The 2θ values of X-ray diffraction peak for Form B are listed in the following table. The characteristic diffraction angles of Form B were about 6.29°, about 13.71°, about 18.20°, about 20.81° and about 22.94°.

| Angle 2θ | Relative Intensity |
|---|---|
| (°) | (%) |
| 4.95 | 17 |
| 6.29 | 100 |
| 7.63 | 21 |
| 9.41 | 11 |
| 11.43 | 7 |
| 12.61 | 29 |
| 13.71 | 58 |
| 14.83 | 21 |
| 16.89 | 28 |
| 18.20 | 83 |
| 18.83 | 58 |
| 19.95 | 64 |
| 20.81 | 70 |
| 22.94 | 39 |

X-ray powder diffraction was measured from 2.5° to 32.5° in 2θ, using Philips MPD1880 X-Ray Powder Diffraction System (Holland). The samples were illuminated with a graphite-monochromated Cu—Kα radiation (λ=1.5418 Å) at 40 mA and 30 kV. The goniometer was equipped with a 1° dispersion slit, a 0.2 mm receiving slit, and a 1° scatter slit. A proportional counter was used for detection.

In the DSC measurement, Form A exhibited an endothermic peak due to melting-decomposition at 224° C. (onset temperature). Form B exhibited a small endothermic at 118° C. (onset temperature) due to melting, followed by an exothermic peak due to thermal recrystallization of Form C at 164° C. (peak top temperature) and an endothermic peak due to melting-decomposition at 219° C. (onset temperature).

DSC6200 (Seiko Instruments, Japan) was used for these DSC measurements. Samples were weighed into aluminum pans (open system, aluminum plate covers were used) and empty pans were used as the reference. And measurements were carried out from room temperature to about 270° C., with a heating rate of 10° C./min, under a nitrogen atmosphere (30 ml/min). A sampling time was 0.2 second.

Preferred embodiments of the object compound [Ig] are as follow:

R¹ is hydrogen,

R² is hydrogen, and

R³ and R⁴ are each N-methyl-methoxycarbonylamino.

The following Preparations and Examples are given for the purpose of illustrating this invention.

Preparation 1

To a mixture of 3,3-diphenylpropylamine (120 g), pyridine (53 ml) and dichloromethane (300 ml), trifluoroacetic anhydride (84 ml) was added dropwise below 10° C. After stirring for 30 minutes, the reaction mixture was added to a mixture of concentrated hydrochloric acid (20 ml) and ice-water (200 ml). The organic phase was separated, washed twice with water, followed by washing with saturated sodium chloride solution, dried over anhydrous magnesium sulfate, filtered and evaporated to afford N-(3,3-diphenylpropyl)trifluoroacetamide (179 g).

Preparation 2

To a cooled mixture of 97% sulfuric acid (8 ml) and 70% nitric acid (7.8 ml), acetic anhydride (15.6 ml) was added dropwise below 20° C. To the reaction mixture, N-(3,3-diphenylpropyl)trifluoroacetamide (powdered, 7.80 g) was added. After stirring at room temperature for 1 hour, the reaction mixture was added to ice-water, followed by addition of ethyl acetate (200 ml). The organic phase was washed successively with water (3 times) and saturated sodium chloride solution (once), dried over anhydrous magnesium sulfate, filtered and evaporated. The crude residue was purified by column chromatography (silica gel, toluene:ethyl acetate=20:1 to 4:1)) to afford N-[3,3-bis(4-nitrophenyl)propyl]trifluoroacetamide (6.46 g).

NMR (CDCl₃, δ): 2.45 (2H, quartet, J=7.3 Hz), 3.35 and 3.39 (2H, each t, J=7.8 Hz), 4.21 (1H, t, J=7.8 Hz), 6.41 (1H, br s), 7.42 (4H, d, J=8.7 Hz), 8.20 (4H, d, J=8.7 Hz)

Preparation 3

A mixture of N-[3,3-bis(4-nitrophenyl)propyl]-trifluoroacetamide (890 mg), iron powder (0.90 g), ammonium chloride (0.10 g), ethanol (9 ml) and water (2 ml) was heated under reflux for 0.5 hour, cooled to room temperature, filtered and evaporated. The residue was dissolved in ethyl acetate, washed with water followed by saturated sodium chloride solution, dried over anhydrous magnesium sulfate, filtered and evaporated to afford N-[3,3-bis(4-aminophenyl)propyl]trifluoroacetamide (774 mg).

NMR (CDCl₃, δ): 2.22 (2H, quartet, J=7.3 Hz), 3.32 and 3.36 (2H, each t, J=6.6 Hz), 3.58 (4H, br s), 3.74 (1H, t, J=7.9 Hz), 6.12 (1H, br s), 6.61 (4H, d, J=8.4 Hz), 6.98 (4H, d, J=8.4 Hz)

MS m/z: 360 (M⁺+Na)

Preparation 4

To a mixture of N-[3,3-bis(4-aminophenyl)propyl]-trifluoroacetamide (729 mg), pyridine (0.52 ml) and dichloromethane (5 ml), methyl chlorocarbonate (0.37 ml) was added below 10° C. After stirring at room temperature overnight, the reaction mixture was washed with water followed by saturated sodium chloride solution, dried over anhydrous magnesium sulfate, filtered and evaporated to afford N-[3,3-bis[4-(methoxycarbonylamino)phenyl]propyl]-trifluoroacetamide (995 mg).

NMR (CDCl₃, δ): 2.29 (2H, quartet, J=7.8 Hz), 3.30 and 3.33 (2H, each t, J=6.5 Hz), 3.76 (6H, s), 3.87 (1H, t, J=7.9 Hz), 6.31 (1H, br s), 6.62 (2H, s), 7.13 (4H, d, J=8.6 Hz), 7.30 (4H, d, J=8.6 Hz)

MS m/z: 454 (M⁺+1)

Preparation 5

A mixture of N-[3,3-bis[4-(methoxycarbonylamino)-phenyl]propyl]trifluoroacetamide (500 mg), methanol (5 ml), 1,4-dioxane (5 ml), potassium carbonate (0.23 g) and water (3 ml) was stirred at 40° C. After 2 hours, potassium carbonate (0.23 g) and water (5 ml) were added to the reaction mixture and stirred at 50° C. for 3 hours. The reaction mixture was extracted with ethyl acetate. The extracted ethyl acetate solution was dried over anhydrous potassium carbonate, filtered and evaporated to afford 3,3-bis[4-(methoxycarbonylamino)phenyl]propylamine (458 mg) as a crude powder.

NMR (CDCl₃, δ): 1.51 (2H, br s), 2.13 (2H, quartet, J=7.6 Hz), 2.64 (2H, t, J=6.9 Hz), 3.75 (6H, s), 3.95 (1H, t, J=7.8 Hz), 6.56 (2H, s), 7.14 (4H, d, J=8.6 Hz), 7.28 (4H, d, J=7.1 Hz)

MS m/z: 358 (M⁺+1)

Preparation 6

A mixture of 3,3-bis[4-(methoxycarbonylamino)phenyl]-propylamine (69.3 g), benzaldehyde (24 ml) and 1,4-dioxane (200 ml) was heated under reflux for 1 hour. To the reaction mixture, sodium borohydride (8.8 g) was added portionally below 10° C., followed by dropwise addition of methanol (40 ml). After the reaction mixture was stirred at room temperature for 1 hour, water (0.5 l) and ethyl acetate (0.5 l) were added to the resulting mixture. The organic phase was separated, washed with water (0.5 l×3 times) followed by saturated sodium chloride solution (0.5 l), dried over anhydrous magnesium sulfate, filtered and evaporated. The crude residue was dissolved in ethyl acetate (200 ml) and therein was added 4N hydrogen chloride ethyl acetate solution (58 ml) dropwise below 10° C. After standing for 20 minutes below 10° C., hexane (200 ml) was added and the mixture was allowed to stand for 30 minutes. The precipitated gummy product was separated by decantation, triturated with diisopropyl ether (300 ml) to afford a powder of hydrochloride of the objective compound. After free basing the powder in a usual manner, N-benzyl-[3,3-bis[4-(methoxycarbonylamino)phenyl]propyl]amine (75.7 g) was obtained.

MS m/z: 448 (M⁺+1)

Preparation 7

To an ice-cooled solution of N-benzyl-[3,3-bis[4-(methoxycarbonylamino)phenyl]propyl]amine (45.7 g) in methanol (120 ml), 4N hydrogen chloride in 1,4-dioxane (21 ml) was added dropwise. The mixture was evaporated to afford an oily residue. The residue was triturated with ethyl acetate (300 ml) to afford a powder, which was collected by filtration, washed with ethyl acetate and dried to afford N-benzyl-[3,3-bis[4-(methoxycarbonylamino)phenyl]-propyl]amine hydrochloride (39.7 g).

NMR (CDCl₃, δ): 2.34-2.47 (2H, m), 2.92-3.00 (2H, m), 3.71 (6H, s), 3.94 (1H, t, J=7.9 Hz), 4.15 (2H, s), 7.18 (4H, d, J=8.6 Hz), 7.36 (4H, d, J=8.6 Hz)

Preparation 8

To a suspension of N-benzyl-[3,3-bis[4-(methoxycarbonylamino)phenyl]propyl]amine hydrochloride (35.6 g) in methanol (100 ml) was added water (100 ml). To the mixture was added sodium hydrogen carbonate (8.4 g) portionwise. After 10 minutes stirring, the precipitate was dissolved. The mixture was partitioned between ethyl acetate and water. The aqueous layer was extracted with ethyl acetate. The combined organic layers were stirred with saturated aqueous solution of sodium hydrogen carbonate at room temperature for 30 minutes. The organic layer was washed with brine, dried over magnesium sulfate and evaporated in vacuo to give N-benzyl-[3,3-bis[4-(methoxycarbonylamino)phenyl]propyl]amine (34.4 g) as a brownish oil which was used for the next reaction without further purification.

Preparation 9

To a suspension of powdered potassium carbonate (3.48 g) in N,N-dimethylformamide (39 ml) were added successively N-benzyl-3,3-bis[4-(methoxycarbonylamino)phenyl]propylamine hydrochloride (4.84 g) and benzyl bromide (1.44 ml) at room temperature. After the slight exothermic reaction was ceased, the mixture was stirred at room temperature for additional 1.5 hours and partitioned between hexane/ethyl acetate (1/1) and water. The organic layer was separated, washed successively with water and brine, dried over magnesium sulfate, and filtered. The filtrate was concentrated and the residue was purified by column chromatography (silica gel, hexane/ethyl acetate) to give N,N-dibenzyl-3,3-bis[4-(methoxycarbonylamino)phenyl]-propylamine (5.30 g) as a pale yellow amorphous powder.

NMR (CDCl₃, δ): 2.05-2.48 (4H, m), 3.52 (4H, s), 3.75 (6H, s), 3.86 (1H, t, J=7 Hz), 6.52 (2H, br s), 7.00 (4H, d, J=8 Hz), 7.10-7.38 (14H, m)

MS m/z: 538 (M⁺+1)

Preparation 10

To a suspension of lithium aluminum hydride (739 mg) in tetrahydrofuran (21 ml) was added dropwise N,N-dibenzyl-3,3-bis[4-(methoxycarbonylamino)phenyl]propylamine (5.24 g) in tetrahydrofuran (58 ml) at room temperature over 10 minutes, and the mixture was heated at 60° C. for 5 hours. After allowed to cool to room temperature, the mixture was cooled with ice. Water (0.75 ml), 15% sodium hydroxide solution (0.75 ml), and water (2.2 ml) were added successively to the cooled mixture with vigorous stirring, and the precipitate formed was removed by filtration. The filtrate was concentrated and the residue was purified by column chromatography (silica gel, hexane/ethyl acetate) to give N,N-dibenzyl-3,3-bis[4-(methylamino)phenyl]propylamine (2.43 g) as a pale yellow oil.

NMR (CDCl₃, δ): 2.06-2.26 (2H, m), 2.34-2.50 (2H, m), 2.78 (6H, s), 3.53 (4H, s), 3.53 (2H, br s), 3.71 (1H, t, J=8 Hz), 6.47 (4H, d, J=8 Hz), 6.93 (4H, d, J=8 Hz), 7.12-7.42 (10H, m)

MS m/z: 450 (M⁺+1)

Preparation 11

To an ice-cooled solution of 3,3-bis(3-aminophenyl)-N,N-dibenzyl-2-propen-1-amine (483 mg) in dichloromethane (2.4 ml) were added pyridine (0.28 ml) and methyl chlorocarbonate (0.21 ml). The mixture was stirred at the same temperature for 1.5 hours and partitioned between ethyl acetate and saturated sodium bicarbonate solution. The organic layer was separated, washed with brine, dried over magnesium sulfate, and filtered. The filtrate was concentrated and the residue was purified by column chromatography (silica gel, hexane/ethyl acetate) to give N,N-dibenzyl-3,3-bis[3-(methoxycarbonylamino)phenyl]-2-propen-1-amine (561 mg) as a white amorphous powder.

NMR (CDCl₃, δ): 3.15 (2H, d, J=7 Hz), 3.57 (4H, s), 3.74 (3H, s), 3.76 (3H, s), 6.22 (1H, t, J=7 Hz), 6.51 (2H, br s), 6.68-6.95 (4H, m), 7.10-7.53 (14H, m) MS m/z: 536 (M⁺+1)

Preparation 12

The following compounds were obtained according to a similar manner to that of Preparation 11.

(1) N,N-Dibenzyl-3,3-bis[4-[N-(methoxycarbonyl)-N-methylamino]phenyl]propylamine

NMR (CDCl₃, δ): 2.12-2.29 (2H, m), 2.32-2.50 (2H, m), 3.24 (6H, s), 3.54 (4H, s), 3.69 (6H, s), 3.93 (1H, t, J=7 Hz), 7.06 (8H, s), 7.14-7.40 (10H, m)

MS m/z: 566 (M⁺+1)

(2) 3-Dibenzylamino-1,1-bis[3-(methoxycarbonylamino)-phenyl]-1-propanol

NMR (CDCl₃, δ): 2.40-2.57 (2H, m), 2.57-2.75 (2H, m), 3.52 (4H, s), 3.75 (6H, s), 6.44 (2H, br s), 6.93 (2H, d, J=8 Hz), 7.04 (2H, s), 7.12 (2H, t, J=8 Hz), 7.18-7.42 (12H, m), 7.75 (12H, br s)

MS m/z: 554 (M⁺+1)

Preparation 13

To a solution of N,N-dibenzyl-3,3-bis[3-(methoxycarbonylamino)phenyl]-2-propen-1-amine (157 mg) in methanol (1.6 ml) was added 4N hydrogen chloride/1,4-dioxane (0.16 ml), and the mixture was hydrogenated (1 atm) over 20% palladium hydroxide on carbon (17 mg) at room temperature for 4.5 hours. The catalyst was filtered off, the filtrate was evaporated, and the residue was partitioned between ethyl acetate and saturated sodium bicarbonate solution. The organic layer was separated, washed with brine, dried over magnesium sulfate, and filtered. The filtrate was evaporated to give N-benzyl-3,3-bis[3-(methoxycarbonyl-amino)phenyl]propylamine (146 mg) as a white amorphous powder.

NMR (CDCl₃, δ): 2.10-2.30 (2H, m), 2.50-2.68 (2H, m), 3.71 (2H, s), 3.74 (6H, s), 3.94 (1H, t, J=8 Hz), 6.79 (2H, br s), 6.85-6.98 (2H, m), 7.08-7.38 (11H, m)

MS m/z: 448 (M⁺+1)

Preparation 14

The following compounds were obtained according to a similar manner to that of Preparation 13.

(1) N-Benzyl-3,3-bis[4-[N-(methoxycarbonyl)-N-methylamino]phenyl]propylamine

NMR (CDCl₃, δ): 2.12-2.33 (2H, m), 2.52-2.70 (2H, m), 3.26 (6H, s), 3.69 (6H, s), 3.73 (2H, s), 4.02 (1H, t, J=8 Hz), 7.02-7.40 (13H, m)

MS m/z: 476 (M⁺+1)

(2) 3-Benzylamino-1,1-bis[3-(methoxycarbonylamino)phenyl]-1-propanol

NMR (CDCl₃, δ): 2.30-2.48 (2H, m), 2.73-2.89 (2H, m), 3.70 (2H, s), 3.75 (6H, s), 6.60 (2H, br s), 7.02-7.48 (13H, m)

MS m/z: 464 (M⁺+1)

(3) N-Benzyl-3,3-bis[4-(3-ethylureido)phenyl]propylamine

NMR (CDCl₃, δ): 1.14 (6H, t, J=7 Hz), 2.05-2.70 (4H, m), 3.25 (4H, q, J=7 Hz), 3.71 (2H, s), 3.90-4.00 (1H, m), 4.60-4.80 (2H, m), 7.00-7.20 (13H, m)

MS m/z: 474 (M⁺+1)

Preparation 15

To an ice-cooled solution of N-benzyl-3,3-bis[4-(methoxycarbonylamino)phenyl]propylamine hydrochloride (4.84 g) in a mixture of 1,4-dioxane (14.5 ml) and 1N sodium hydroxide solution (11.5 ml) was added dropwise di-tert-butyl dicarbonate (2.32 g) in 1,4-dioxane (4.8 ml) over 5 minutes. The mixture was stirred at room temperature for 1.5 hours before partitioned between ethyl acetate and water. The organic layer was separated, washed successively with water and brine, dried over magnesium sulfate, and filtered. The filtrate was concentrated and the residue was purified by column chromatography (silica gel, hexane/ethyl acetate) to give N-benzyl-N-(tert-butoxycarbonyl)-3,3-bis[4(methoxycarbonylamino)phenyl]propylamine (5.66 g) as a pale yellow amorphous powder.

NMR (CDCl₃, δ): 1.44 (9H, s), 2.05-2.29 (2H, m), 2.94-3.22 (2H, m), 3.70 (1H, m), 3.75 (6H, s), 4.37 (2H, br s), 6.62 (2H, br s), 6.98-7.37 (13H, m)

MS m/z: 570 (M++Na)

Preparation 16

To an ice-cooled suspension of sodium hydride (60% in mineral oil, 175 mg) in N,N-dimethylformamide (1 ml) was added dropwise N-benzyl-N-(tert-butoxycarbonyl)-3,3-bis[4-(methoxycarbonylamino)phenyl]propylamine (1.09 g) in N,N-dimethylformamide (4.4 ml) over 5 minutes, and the mixture was stirred at room temperature for 30 minutes before cooled with ice. Iodoethane (0.40 ml) was added to the cooled mixture, and the resulting suspension was stirred at room temperature for 1.5 hours and partitioned between hexane/ethyl acetate (1/1) and water. The organic layer was separated, washed successively with water and brine, dried over magnesium sulfate, and filtered. The filtrate was concentrated to give N-benzyl-N-(tert-butoxycarbonyl)-3,3-bis[4-[N-ethyl-N-(methoxycarbonyl)amino]phenyl]propylamine (1.29 g) as an oil.

NMR (CDCl₃, δ): 1.13 (6H, t, J=7 Hz), 1.44 (9H, s), 2.10-2.38 (2H, m), 2.96-3.26 (2H, m), 3.67 (6H, s), 3.67 (4H, q, J=7 Hz), 3.70-3.93 (1H, m), 4.22-4.48 (2H, m), 7.02-7.22 (8H, m), 7.22-7.36 (5H, m)

MS m/z: 626 (M⁺+Na)

Preparation 17

The following compound was obtained according to a similar manner to that of Preparation 16.

N-Benzyl-N-(tert-butoxycarbonyl)-3,3-bis [4-[N-methoxycarbonyl)-N-propylamino]phenyl]propylamine

NMR (CDCl₃, δ): 0.87 (6H, t, J=7 Hz), 1.39-1.67 (4H, m), 1.44 (9H, s), 2.10-2.38 (2H, m), 2.96-3.24 (2H, m), 3.48-3.66 (4H, m), 3.66 (6H, s), 3.66-3.94 (1H, m), 4.22-4.49 (2H, br s), 6.98-7.39 (13H, m)

MS m/z: 654 (M⁺+Na)

Preparation 18

To an ice-cooled solution of N-benzyl-N-(tert-butoxycarbonyl)-3,3-bis[4-[N-ethyl-N-(methoxycarbonyl)-amino]phenyl]propylamine (1.18 g) in dichloromethane (1.2 ml) was added 4N hydrogen chloride/1,4-dioxane (2.5 ml), and the mixture was stirred at room temperature for 1.5 hours. The mixture was concentrated, and the residue was partitioned between ethyl acetate and saturated sodium bicarbonate solution. The organic layer was separated, washed successively with water and brine, dried over magnesium sulfate, and filtered. The filtrate was concentrated to give N-benzyl-3,3-bis[4-[N-ethyl-N-(methoxycarbonyl)amino]phenyl]propylamine (1.13 g) as an oil.

NMR (CD₃OD, 6): 1.13 (6H, t, J=7 Hz), 2.14-2.36 (2H, m), 2.52-2.70 (2H, m), 3.67 (6H, s), 3.67 (4H, q, J=7 Hz), 3.74 (2H, s), 4.04 (1H, t, J=8 Hz), 6.98-7.40 (13H, m)

MS m/z: 504 (M⁺+1)

Preparation 19

The following compound was obtained according to a similar manner to that of Preparation 18.

N-Benzyl-3,3-bis[4-[N-(methoxycarbonyl)-N-propylamino]phenyl]propylamine

NMR (CDCl₃, δ): 0.87 (6H, t, J=7 Hz), 1.39-1.67 (4H, m), 2.12-2.36 (2H, m), 2.50-2.92 (2H, m), 3.46-3.68 (4H, m), 3.66 (6H, s), 3.74 (2H, s), 4.03 (1H, t, J=8 Hz), 6.96-7.40 (13H, m)

MS m/z: 532 (M⁺+1)

Preparation 20

To a solution of 1,3-dibromobenzene (10.38 g) in tetrahydrofuran (88 ml) was added dropwise 1.54 M butyllithium/hexane (27 ml) at −70° C. over 45 minutes. The resulting suspension was allowed to warm to about −20° C. before cooled to −70° C. again. To the suspension was added dropwise ethyl 3-(dibenzylamino)propionate (5.95 g) in tetrahydrofuran (12 ml) at about −65° C. over 10 minutes. The mixture was stirred at −70° C. for 1.5 hours and allowed to warm to room temperature for 1.5 hours. The mixture was partitioned between ethyl acetate and water. The organic layer was separated, washed successively with water and brine, dried over magnesium sulfate, and filtered. The filtrate was concentrated and the residue was purified by column chromatography (silica gel, hexane/ethyl acetate) to give 3-dibenzylamino-1,1-bis(3-bromophenyl)-1-propanol (9.38 g) as an oil.

NMR (CDCl₃, δ): 2.28-2.48 (2H, m), 2.58-2.76 (2H, m), 3.52 (4H, s), 6.90-7.52 (18H, m)

MS m/z: 564, 566, 568 (M⁺+1)

Preparation 21

A mixture of 3-dibenzylamino-1,1-bis(3-bromophenyl)-1-propanol (2.88 g) and p-toluenesulfonic acid monohydrate (2.89 g) in toluene (23 ml) was heated to reflux for 2.5 hours. After allowed to cool to room temperature, the mixture was neutralized with saturated sodium bicarbonate solution and extracted twice with ethyl acetate. The combined extract was washed successively with water and brine, dried over magnesium sulfate, and filtered. The filtrate was concentrated and the residue was purified by column chromatography (silica gel, hexane/ethyl acetate) to give N,N-dibenzyl-3,3-bis(3-bromophenyl)-2-propen-1-amine (2.38 g) as an oil.

NMR (CDCl₃, δ): 3.12 (2H, d, J=7 Hz), 3.57 (4H, s), 6.21 (1H, t, J=7 Hz), 6.90-7.48 (18H, m)

MS m/z: 546, 548, 550 (M⁺+1)

Preparation 22

A mixture of N,N-dibenzyl-3,3-bis(3-bromophenyl)-2-propen-1-amine (2.36 g), benzophenone imine (1.89 g), tris(dibenzylideneacetone)dipalladium(0) (200 mg), racemic 2,2′-bis(diphenylphosphino)-1,1′-binaphthyl (318 mg), and sodium tert-butoxide (906 mg) in toluene (11 ml) was heated at 80° C. for 1 hour. The mixture was partitioned between ethyl acetate and water. The organic layer was separated, washed with brine, dried over magnesium sulfate, and filtered. The filtrate was concentrated and the residual red-brown oil was dissolved in tetrahydrofuran (12 ml). To the solution was added 6N hydrochloric acid (4.3 ml), and the mixture was stirred at room temperature for 2.5 hours. The mixture was neutralized with saturated sodium bicarbonate solution and extracted twice with chloroform. The combined extract was washed with brine, dried over magnesium sulfate, and filtered. The filtrate was concentrated and the residue was purified by column chromatography (silica gel, chloroform/ethyl acetate) to give 3,3-bis(3-aminophenyl)-N,N-dibenzyl-2-propen-1-amine (964 mg) as a pale yellow solid.

NMR (CDCl₃, δ): 3.15 (2H, d, J=7 Hz), 3.56 (4H, s), 3.56 (4H, br s), 6.17 (1H, t, J=7 Hz), 6.30-6.72 (6H, m), 6.93-7.48 (12H, m)

MS m/z: 420 (M⁺+1)

Preparation 23

The following compound was obtained according to a similar manner to that of Preparation 22.

1,1-Bis(3-aminopheyl)-3-dibenzylamino-1-propanol

NMR (CDCl₃, δ): 2.32-2.50 (2H, m), 2.56-2.78 (2H, m), 3.48 (4H, br s), 3.54 (4H, s), 6.44 (2H, dd, J=8 and 2 Hz), 6.59-6.75 (4H, m), 6.96 (2H, t, J=8 Hz), 7.12-7.44 (10H, m)

MS m/z: 438 (M⁺+1)

Preparation 24

To a solution of 1,1-bis(4-aminophenyl)-3-(dibenzylamino)-1-propanol (500 mg) in tetrahydrofuran (5.0 ml) was added ethyl isocyanate (0.23 ml) dropwise under ice-water cooling. The mixture was stirred at the same temperature for 1 hour and at room temperature for 2 days. The reaction mixture was evaporated in vacuo. The residue was purified by a silica gel column chromatography (chloroform-ethyl acetate) to give 1,1-bis[4-(3-ethylureido)phenyl]-3-dibenzylamino-1-propanol (566 mg) as a brownish amorphous powder.

NMR (CDCl₃, δ): 1.16 (6H, t, J=6 Hz), 2.36-2.70 (4H, m), 3.14-3.36 (4H, m), 3.54 (4H, s), 4.70 (2H, s), 6.20 (2H, s), 6.96-7.36 (18H, m)

Preparation 25

A mixture of N-benzyl-[3,3-bis[4-(methoxycarbonylamino)phenyl]propyl]amine (33 g), (S)-2-(phenoxymethyl)oxirane (11.1 g) and 2-propanol (222 ml) was heated at 90° C. for 13 hours and evaporated. The residue was purified by flush column chromatography (silica gel, hexane:ethyl acetate=3:1 to 1:1) to afford (2S)-1-phenoxy-3-[N-benzyl-[3,3-bis[4-(methoxycarbonylamino)phenyl]propyl]-amino]-2-propanol (43.1 g).

NMR (CDCl₃, δ): 2.1-2.3 (2H, m), 2.4-2.7 (4H, m), 3.51 (1H, d, J=13.4 Hz), 3.75 (6H, s), 3.76 (1H, d, J=13.4 Hz), 3.8 (1H, m), 3.9 (1H, m), 3.93 (2H, s), 6.53 (12H, s), 6.86 (2H, d, J=8.0 Hz), 6.96 (1H, d, J=8.0 Hz), 7.06 (4H, m), 7.2-7.4 (11H, m)

MS m/z: 598 (M⁺+1)

Preparation 26

A mixture of (R)-2-(phenoxymethyl)oxirane (47 mg), N-benzyl-3,3-bis[4-(methoxycarbonylamino)phenyl]propylamine (140 mg) and ethanol (2.0 ml) was heated under reflux for 18 hours. To the reaction mixture was added 10% palladium on carbon (50 mg). The mixture was stirred at room temperature under hydrogen atmosphere for 6 hours. The mixture was filtered through Celite® and washed with ethanol. The filtrate and washings were combined and evaporated in vacuo. The residue was purified by a silica gel column chromatography (chloroform/methanol) to give (2R)-1-phenoxy-3-[3,3-bis [4-(methoxycarbonylamino)phenyl]propylamino]-2-propanol (77 mg) as a white amorphous powder.

NMR (CDCl₃, δ): 2.12-2.30 (2H, m), 2.50-2.82 (4H, m), 3.72 (6H, s), 3.84-4.10 (4H, m), 6.84-6.96 (3H, m), 7.14-7.38 (10H, m)

Preparation 27

(2S)-1-Phenoxy-3-[3,3-bis[4-(methoxycarbonylamino)-phenyl]propylamino]-2-propanol (50.7 mg) was dissolved in a solution of sulfuric acid (9.81 mg) in ethanol (0.86 ml) and the resulting solution was evaporated in vacuo. The oily residue was powdered from a mixture of hexane and diisopropyl ether to afford (2S)-1-phenoxy-3-[3,3-bis[4-(methoxycarbonylamino)phenyl]propylamino]-2-propanol sulfate (1:1) (45 mg) as a colorless powder.

NMR (CD₃OD, δ): 2.35-2.55 (2H, m), 2.95-3.05 (2H, m), 3.1-3.25 (2H, m), 3.71 (6H, s), 3.91-4.1 (3H, m), 4.16-4.23 (1H, m), 6.90-6.98 (3H, m), 7.18-7.31 (6H, m), 7.37 (4H, d, J=8.5 Hz)

Preparation 28

(2S)-1-Phenoxy-3-[3,3-bis[4-(methoxycarbonylamino)-phenyl]propylamino]-2-propanol (50.7 mg) and (S)-(+)-mandelic acid (15.2 mg) was dissolved in ethyl acetate and the resulting solution was evaporated in vacuo. The oily residue was powdered from diisopropyl ether to afford (2S)-1-phenoxy-3-[3,3-bis[4-methoxycarbonylamino)phenyl]-propylamino]-2-propanol (S)-mandelate (1:1) (61 mg) as a powder.

NMR (CD₃OD, δ): 2.25-2.5 (2H, m), 2.9-3.0 (2H, m), 3.05-3.3 (2H, m), 3.71 (6H, s), 3.90-4.04 (3H, m), 4.07-4.20 (1H, m), 4.59 (1H, br s), 6.86-6.98 (3H, m), 7.16-7.48 (15H, m)

Preparation 29

(2S)-1-Phenoxy-3-[3,3-bis[4-(methoxycarbonylamino)-phenyl]propylamino]-2-propanol (R)-mandelate (1:1) (60 mg, powder) was prepared from (2S)-1-phenoxy-3-[3,3-bis[4-(methoxycarbonylamino)phenyl]propylamino]-2-propanol (50.7 mg) and (R)-(−)-mandelic acid (15.2 mg) in a similar manner to that of Preparation 28.

NMR (CD₃OD, δ): 2.25-2.5 (2H, m), 2.9-3.0 (2H, m), 3.05-3.3 (2H, m), 3.71 (6H, s), 3.91-4.04 (3H, m), 4.07-4.19 (1H, m), 4.60 (1H, br s), 6.90-6.98 (3H, m), 7.17-7.48 (15H, m)

Preparation 30

To a solution of (2S)-1-phenoxy-3-[3,3-bis[4-(methoxycarbonylamino)phenyl]propylamino]-2-propanol (504 mg) in ethanol (5 ml) was added citric acid (69 mg) at room temperature. The solution was evaporated in vacuo to give a colorless oil. The residue was dissolved in hot ethanol (3.0 ml). Water (6.0 ml) was added to the ethanol solution in a boiling bath to give a slightly clouded hot mixture. The mixture was stirred under ice-water cooling for 3 hours. The precipitate was collected by vacuum filtration and washed with cold ethanol-water (2:1) and then cold water to give a hygroscopic powder. The wet mass was dried in vacuo at room temperature for 1 week to give (2S)-1-phenoxy-3-[3,3-bis[4-(methoxycarbonylamino)phenyl]propylamino]-2-propanol citrate (3:1) as a white powder (298 mg).

NMR (CD₃OD, δ): 2.16-2.50 (2H, m), 2.68 (2/3H, d, J=15 Hz), 2.78 (2/3H, d, J=15 Hz), 2.86-3.24 (4H, m), 3.70 (6H, s), 3.86-4.30 (4H, m), 6.86-7.00 (3H, m), 7.14-7.40 (10H, m)

Preparation 31

To a solution of (2S)-1-phenoxy-3-[3,3-bis[4-(methoxycarbonylamino)phenyl]propylamino]-2-propanol in ethanol was added 1 equivalent of phosphoric acid (85% solution in water) and the mixture was stirred at room temperature for 1 hour. The solvent was removed by evaporation and the residual viscous oil was dissolved in ethyl acetate (1 volume/g). To the solution was added hexane (2 volume/g) to give a crude salt as a paste. The paste was collected by decantation of the solvent, triturated with hexane several times, and dried under reduced pressure to give (2S)-1-phenoxy-3-[3,3-bis[4-(methoxycarbonylamino)phenyl]propylamino]-2-propanol phosphate (1:1) as a white powder.

NMR (CD₃OD, δ): 2.41-2.49 (2H, m), 2.91-3.26 (4H, m), 3.71 (6H, s), 3.91-3.99 (3H, m), 4.17-4.23 (1H, m), 6.88-6.97 (3H, m), 7.18-7.47 (10H, m)

### EXAMPLE 1

A mixture of N-benzyl-3,3-bis[3-(methoxycarbonylamino)-phenyl]propylamine (118 mg) and (S)-2-(phenoxymethyl)oxirane (56 mg) in ethanol (1.2 ml) was heated under reflux for 2 hours. After allowed to cool to room temperature, the mixture was concentrated and the residue was purified by column chromatography (silica gel, hexane/ethyl acetate) to give (2S)-1-phenoxy-3-[N-benzyl-3,3-bis[3-(methoxycarbonylamino)phenyl]propylamino]-2-propanol (119 mg) as a white amorphous powder.

NMR (CDCl₃, δ): 2.06-2.36 (2H, m), 2.36-2.73 (4H, m), 3.52 (1H, d, J-13 Hz), 3.75 (6H, s), 3.76 (1H, d, J=13 Hz), 3.78-4.02 (4H, m), 6.57 (2H, s), 6.73-7.38 (18H, m)

MS m/z: 598 (M⁺+1)

### EXAMPLE 2

The following compounds were obtained according to a similar manner to that of Example 1.

(1) (2S)-1-Phenoxy-3-[N-benzyl-3,3-bis[4-[N-(methoxycarbonyl)-N-methylamino]phenyl]propylamino]-2-propanol

NMR (CDCl₃, δ): 2.10-2.72 (6H, m), 3.25 (6H, s), 3.53 (1H, d, J=13 Hz), 3.69 (6H, s), 3.76 (1H, d, J=13 Hz), 3.82-4.00 (4H, m), 6.78-7.38 (18H, m)

MS m/z: 626 (M⁺+1)

(2) (2S)-1-Phenoxy-3-[N-benzyl-3,3-bis[4-[N-ethyl-N-(methoxycarbonyl)amino]phenyl]propylamino]-2-propanol

NMR (CDCl₃, δ): 1.12 (6H, t, J=7 Hz), 2.09-2.38 (2H, m), 2.38-2.75 (4H, m), 3.53 (1H, d, J=13 Hz), 3.66 (6H, s), 3.66 (4H, q, J=7 Hz), 3.76 (1H, d, J=13 Hz), 3.79-4.00 (4H, m), 6.80-7.38 (18H, m)

MS m/z: 676 (M⁺+Na)

(3) (2S)-1-Phenoxy-3-[N-benzyl-3,3-bis[4-[N-(methoxycarbonyl)-N-propylamino]phenyl]propylamino]-2-propanol

NMR (CDCl₃, δ): 0.87 (6H, t, J=7 Hz), 1.40-1.66 (4H, m), 2.08-2.78 (6H, m), 3.42-4.02 (10H, m), 3.66 (6H, s), 6.79-7.40 (18H, m)

MS m/z: 682 (M⁺+1)

(4) (2S)-1-Phenoxy-3-[N-benzyl-3-hydroxy-3,3-bis[3-(methoxycarbonylamino)phenyl]propylamino]-2-propanol

NMR (CDCl₃, δ): 2.40-2.63 (4H, m), 2.67-2.86 (2H, m), 3.48-3.90 (5H, m), 3.76 (6H, s), 6.57 (2H, br s), 6.68-7.40 (18H, m), 7.52 (1H, br s)

MS m/z: 614 (M⁺+1)

(5) (2S)-1-Phenoxy-3-[N-benzyl-3,3-bis[4-(3-ethylureido)-phenyl]propylamino]-2-propanol

NMR (CDCl₃, δ): 1.08 (6H, t, J=7 Hz), 1.98-2.66 (6H, m), 3.10-3.33 (4H, m), 3.47 (1H, d, J=13 Hz), 3.68-3.98 (4H, m), 3.72 (1H, d, J=13 Hz), 5.29 (2H, br s), 6.73-7.34 (18H, m)

MS m/z: 624 (M⁺+1)

### EXAMPLE 3

A solution of (2S)-1-phenoxy-3-[N-benzyl-3,3-bis[3-(methoxycarbonylamino)phenyl]propylamino]-2-propanol (105 mg) in methanol (2.1 ml) was hydrogenated (1 atm) over 10% palladium on carbon (15 mg) at room temperature for 24 hours.

After the catalyst was filtered off, the filtrate was concentrated and the residue was purified by column chromatography (silica gel, chloroform/methanol) to give (2S)-1-phenoxy-3-[3,3-bis[3-(methoxycarbonylamino)phenyl]-propylamino]-2-propanol (77 mg) as a white amorphous powder.

IR (Neat): 1710, 1600, 1548, 1492, 1446, 1242 cm⁻¹

NMR (CDCl₃, δ): 2.06-2.40 (2H, m), 2.47-2.94 (4H, m), 3.76 (6H,s), 3.78-4.21 (4H, m), 6.66-7.48 (15H, m)

MS m/z: 508 (M⁺+1)

### EXAMPLE 4

The following compounds were obtained according to a similar manner to that of Example 3.

(1) (2S)-1-Phenoxy-3-[3,3-bis[4-[N-(methoxycarbonyl)-N-methylamino]phenyl]propylamino]-2-propanol

NMR (CDCl₃, δ): 2.12-2.42 (4H, m), 2.55-2.89 (3H, m), 3.26 (6H, s), 3.69 (6H, s), 3.85-4.12 (3H, m), 6.83-7.02 (2H, m), 7.06-7.36 (11H, m)

MS m/z: 536 (M⁺+1)

(2) (2S)-1-Phenoxy-3-[3,3-bis[4-[N-ethyl-N-(methoxycarbonyl)amino]phenyl ]propylamino]-2-propanol

NMR (CDCl₃, δ): 1.13 (6H, t, J=7 Hz), 2.16-2.37 (2H, m), 2.57-2.93 (4H, m), 3.67 (6H, s), 3.67 (4H, q, J=7 Hz), 3.87-4.12 (4H, m), 6.81-7.39 (13H, m)

MS m/z: 564 (M⁺+1)

(3) (2S)-1-Phenoxy-3-[3,3-bis[4-[N-(methoxycarbonyl)-N-propylamino]phenyl]propylamino]-2-propanol

NMR (CDCl₃, δ): 0.87 (6H, t, J=7 Hz), 1.41-1.67 (4H, m), 2.15-2.37 (2H, m), 2.56-2.92 (4H, m), 3.48-3.66 (4H, m), 3.67 (6H, s), 3.88-4.10 (4H, m), 6.81-7.38 (13H, m)

MS m/z: 592 (M⁺+1)

(4) (2S)-1-Phenoxy-3-[3-hydroxy-3,3-bis[3-(methoxycarbonylamino)phenyl]propylamino]-2-propanol

IR (KBr): 1710, 1602, 1548, 1492, 1442, 1238 cm⁻¹

NMR (CDCl₃, δ): 2.24-2.60 (2H, m), 2.60-2.95 (4H, m), 3.70 (6H, s), 3.75-4.18 (3H, m), 6.76-7.50 (15H, m)

MS m/z: 524 (M⁺+1)

(5) (2S)-1-Phenoxy-3-[3,3-bis[4-(3-ethylureido)phenyl]-propylamino]-2-propanol

IR (KBr): 1658 cm⁻¹

NMR (CD₃OD, δ): 1.13 (6H, t, J=7 Hz), 2.10-2.50 (2H, m), 2.71-3.11 (4H, m), 3.20 (4H, q, J=7 Hz), 3.80-4.22 (4H, m), 6.82-7.40 (13H, m)

MS m/z: 534 (M⁺+1)

### EXAMPLE 5

Preparation of crystal Form A of (2S)-1-phenoxy-3-[3,3-bis[4-(methoxycarbonylamino)phenyl]propylamino]-2-propanol sulfate (2:1)

A mixture of (2S)-1-phenoxy-3-[N-benzyl-[3,3-bis[4-(methoxycarbonylamino)phenyl]propyl]amino]-2-propanol (94.71 g), methanol (1 l) and wet 10% palladium on charcoal (10 g) was stirred under hydrogen (1 atm) at room temperature for 2 hours, filtered and evaporated to afford (2S)-1-phenoxy-3-[3,3-bis[4-(methoxycarbonylamino)phenyl]propylamino]-2-propanol as a crude product. To an ethanol (200 ml) solution of the crude product, an ethanol (64 ml) solution of 96% sulfuric acid (8.1 g) was added dropwise below 10° C. The reaction mixture was stirred at room temperature overnight to precipitate colorless crystals, which were collected by filtration (The filtrated solution was used in Example 6.), washed with ethanol and dried to afford crystal Form A of (2S)-1-phenoxy-3-[3,3-bis[4-(methoxycarbonyl-amino)phenyl]propylamino]-2-propanol sulfate (2:1) (54.9 g).

NMR (CD₃OD, δ): 2.4-2.6 (2H, m), 2.9-3.0 (2H, m), 3.0-3.2 (2H, m), 3.70 (6H, s), 3.9-4.0 (3H, m), 4.2-4.4 (1H, m), 6.88-6.95 (3H, m), 7.16-7.37 (10H, m)

### EXAMPLE 6

Preparation of crystal Form B of (2S)-1-phenoxy-3-[3,3-bis[4-(methoxycarbonylamino)phenyl]propylamino]-2-propanol sulfate (2:1)

The filtrated solution in Example 5 was evaporated to afford an oily residue which was dissolved in acetone (80 ml). The resulting solution was stirred at room temperature for 1.5 hours to precipitate colorless crystals, which were collected by filtration, washed with acetone and dried to afford crystal Form B of (2S)-1-phenoxy-3-[3,3-bis[4-(methoxycarbonylamino)phenyl]propylamino]-2-propanol sulfate (2:1) (3.0 g).

Acetone solvate of (2S)-1-phenoxy-3-[3,3-bis[4-(methoxycarbonylamino)phenyl]propylamino]-2-propanol sulfate (2:1) was obtained in a process of preparation of the crystal Form B.

### DESCRIPTION OF THE FIGURES

FIG. 1 is an XPD pattern for crystal Form A of compound [Is]

FIG. 2 is an XPD pattern for crystal Form B of compound [Is]

FIG. 3 is a DSC curve for crystal Form A of compound [Is]

FIG. 4 is a DSC curve for crystal Form B of compound [Is]

FIG. 5 is an XPD pattern for crystal Form D of compound [Is]

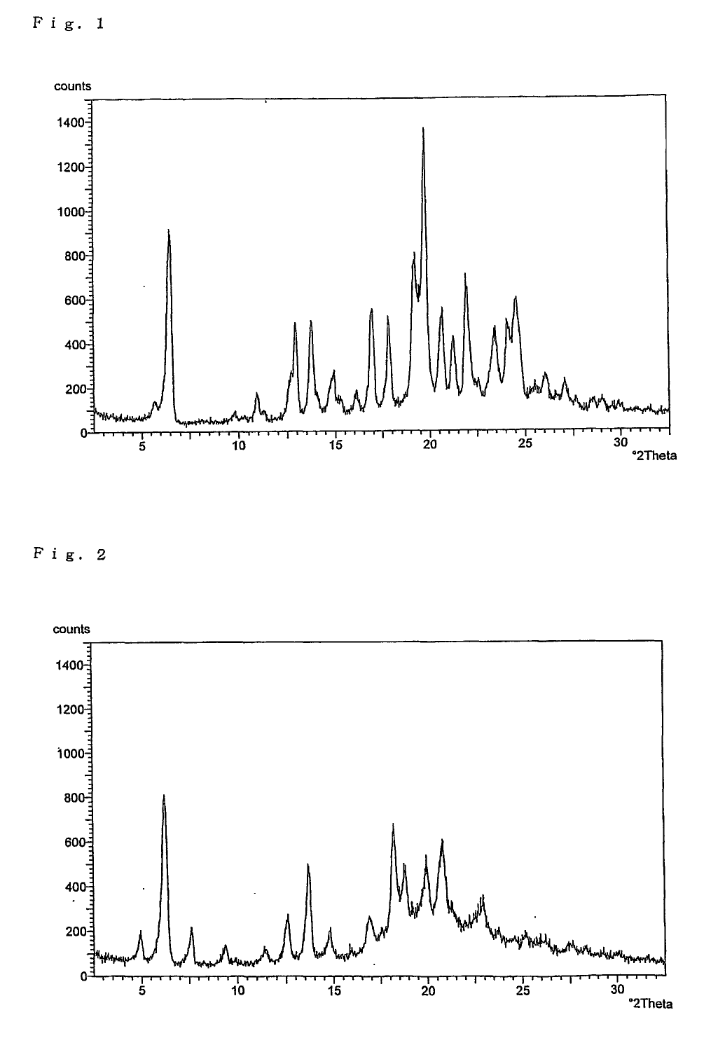

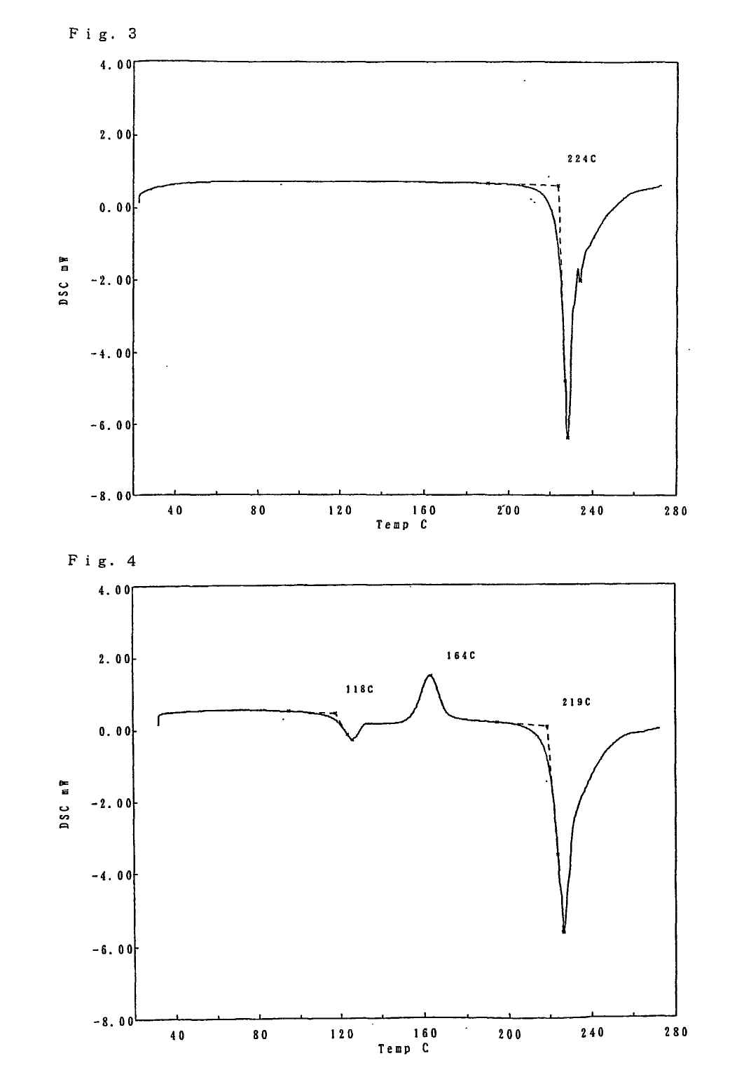

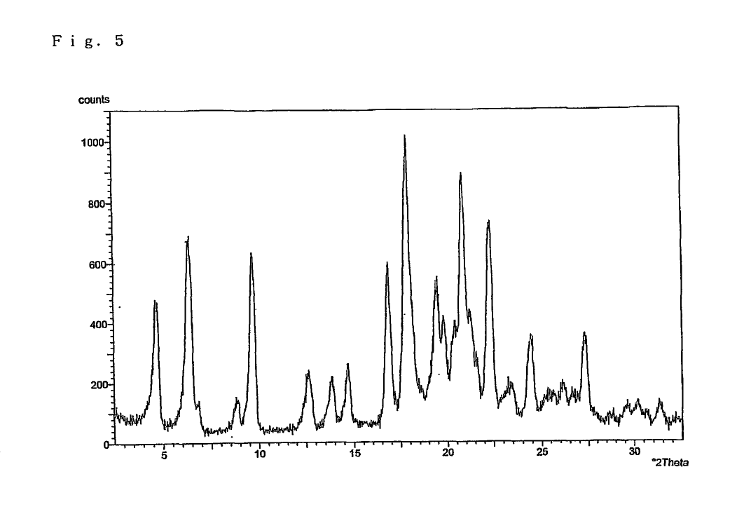

1. A compound of the formula [Is]: 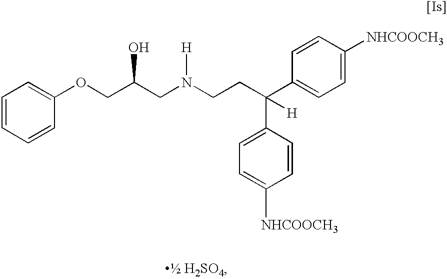
(2S)-1-Phenoxy-3-[3,3-bis[4-(methoxycarbonylamino)-phenyl]propylamino]-2-propanol sulfate (2:1).

2. Crystalline (2S)-1-phenoxy-3-[3,3-bis[4-(methoxy-carbonylamino)phenyl]propylamino]-2-propanol sulfate (2:1).

3. A crystal Form A of (2S)-1-phenoxy-3-[3,3-bis[4-(methoxycarbonylamino)phenyl]propylamino]-2-propanol sulfate (2:1) which shows the peaks at the diffraction angles shown in the following in its X-ray powder diffractometry pattern:
characteristic diffraction angle 2θ (°): about 6.51, about 13.80, about 16.97, about 19.81, about 21.95 and about 24.56.

4. A crystal Form B of (2S)-1-phenoxy-3-[3,3-bis[4-(methoxycarbonylamino)phenyl]propylamino]-2-propanol sulfate (2:1) which shows the peaks at the diffraction angles shown in the following in its X-ray powder diffractometry pattern:
characteristic diffraction angle 2θ (°): about 6.29, about 13.71, about 18.20, about 20.81 and about 22.94.

5. A crystal Form D of (2S)-1-phenoxy-3-[3,3-bis[4-(methoxycarbonylamino)phenyl]propylamino]-2-propanol sulfate (2:1) which shows the peaks at the diffraction angles shown in the following in its X-ray powder diffractometry pattern:
characteristic diffraction angle 2θ (°): about 6.41, about 9.70, about 16.85, about 17.93, about 20.82 and about 22.25.

6. A process for preparing the compound of claim 1, which comprises reacting a compound of (2S)-1-phenoxy-3-[3,3-bis[4-(methoxycarbonylamino)phenyl]propylamino]-2-propanol or a salt thereof other than sulfate thereof with sulfuric acid.

7. A compound of the formula [Ig]: 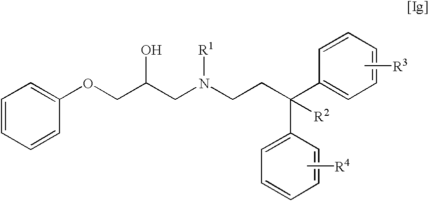
wherein
R¹ is hydrogen or an amino protective group,
R² is hydrogen or hydroxy, and
R³ and R⁴ are independently N-methyl-methoxycarbonylamino, N-ethyl-methoxycarbonylamino, N-propyl-methoxycarbonylamino or 3-ethylureido, or
R³ and R⁴ are both methoxycarbonylamino substituted at a meta position of the benzene rings,
or a salt thereof.

8. A pharmaceutical composition which comprises, as an active ingredient, a compound of claim 1 to 5 and 7 or a pharmaceutically acceptable salt thereof in admixture with pharmaceutically acceptable carriers or excipients.

9. Use of a compound of claim 1 to 5 and 7 or a pharmaceutically acceptable salt thereof for the manufacture of a medicament.

10. A compound of claim 1 to 5 and 7 or a pharmaceutically acceptable salt thereof for use as a medicament.

11. A method for the prophylactic and/or therapeutic treatment of pollakiuria, urinary incontinence, obesity or diabetes, which comprises administering a compound of claim 1 to 5 and 7 or a pharmaceutically acceptable salt thereof to a human being or an animal.
